(19) United States
(12) Patent Application Publication
(10) Pub. No.: US 2008/0058604 A1
(43) Pub. Date: Mar. 6, 2008

(54) CHILDBIRTH LABOR REDUCTION DEVICE

(76) Inventor: Bradford Tyler Sorensen, Manhattan Beach, CA (US)

Correspondence Address: BRADFORD T. SORENSEN, 515 13TH STREET, MANHATTAN BEACH, CA, 90266, US

(21) Appl. No.: 11/469,082

(22) Filed: Aug. 31, 2006

(51) Int. Cl. A61B 1/32

(52) U.S. Cl. 600/208

(57) ABSTRACT

The present invention is a less painful and faster device and method for delivering a baby, which gently and safely opens the birth canal and cervix, forming an expanding tunnel, which enlarges over a period of time, to become large enough in diameter for the baby's head and body to slide out of, for easier, quicker childbirth.

### BACKGROUND AND SUMMARY OF THE INVENTION

The present invention relates to a less painful and faster method of delivering a baby.

Specifically, the current invention provides a device that gently and safely opens the birth canal and cervix, forming an expanding tunnel, which enlarges over a relatively short period of time, to become large enough in diameter for the baby's head and body to slide out of, for faster, easier childbirth.

Childbirth has historically been an extremely painful and exhausting trauma for women to endure. Generally, childbirth can take a couple of hours to a couple of days of painful contractions resulting in the compression of the baby's head and body down into the opening of the pelvis bones, compressing the delicate tissue of the birth canal through the opening first, then effacing or turning the delicate tissue inside out under the painful pressure of the baby's relentlessly advancing head and body until the strength of the contractions contort and stretch the tissue to expel the head and body in an excruciating experience of pain. Childbirth is so painful; some women choose to have powerful drugs injected into their spinal columns to reduce the pain. Childbirth is so painful; some women choose a Caesarian Section Operation to avoid the trauma of childbirth. Childbirth is so painful; some women have themselves rendered unconscious to avoid the pain. Childbirth is so painful, traumatic and exhausting; some women with complicating factors die during childbirth. The current invention provides an alternative to the pain and exhaustion of childbirth.

Historically, doctors have used the Caesarian Section Operation for a significant number of breach births and other problematic births such as severe cephalo-pelvic disproportion, placenta previa, vaso previa, and other contraindications to vaginal delivery or the head, descending too slowly. The current invention opens up a tunnel for the baby to slide out of whether the baby is head first in a normal birth, feet first in a breach birth, or placenta first in a condition called placenta previa, or when the cord blood vessels present themselves across the cervical Os in a condition called vaso previa.

Historically, doctors have used an episiotomy operation on pregnant women delivering babies. Cutting the stretched skin between the vagina and the rectum to allow the baby out is the nightmare of episiotomy. Cutting a slot in a woman's stretched, delicate, sensitive, vagina and rectum just before the baby is given to the mother for the first time is a trauma of profound proportion. The act of stitching up the bleeding episiotomy wound, while the mother is holding the newborn baby in her arms for the first time is a major physical and emotional trauma. Episiotomy operation scars often become infected because of their location in the rectum and perforations in the episiotomy wounds can cause a transfer of body fluids from the rectum into the vagina. The current invention provides an alternative to the use of an episiotomy to remove a baby from its mother. The current invention reduces the need for episiotomy operations by gently and safely opening up the cervix and birth canal, up inside the body cavity where there is more room, instead of allowing the cervix and birth canal to be pushed down, by the baby's head and body during contractions, to be stretched out into the bones of the pelvis where they are painfully turned inside out and stretched to their tearing strength limits and beyond. The current invention significantly reduces the need for episiotomy operations.

Historically forceps are used to apply traction to the fetal head in order to assist childbirth. Historically, Sanskrit writings from approximately 1500 BC contain evidence of single and paired instruments for delivering babies. Peter Chamberlen (drca 1600 England) is given credit for the invention of the precursor of the modem forceps. Modifications have led to more that 700 different types and shapes of forceps.

Obstetrical forceps are typically, in their various types two bladed affairs, which are blindly inserted one blade at a time in a hopefully temporal-cheek position and then articulated together before birth assisting traction is applied. Actual traction is exerted slightly below or underneath the mandibles. The traction is pressure point concentrated and slippage of the forceps is increased because of natural lubrication. Even proper positioning of the forceps can result in harm to the fetus. For example, in instances of minimal cephalo-pelvic disproportion, the insertion of one blade of the forceps can exacerbate any slight deficiency in birth canal adequacy. In addition the softness, or pliability, of the fetal skull, coupled with the existence of sutures which separate the plates of the skull, render the skull susceptible to trauma associated with metal forceps assisted deliveries. The current invention opens up a tunnel for the baby to slide out of so the use of forceps for vaginal delivery is eliminated.

Historically women have demanded powerful pain management drugs, epidurals and/or spinal blocks to reduce the pain of childbirth. A Mother meeting her child for the first time is no time to be drugged into dull, insensitivity. The current invention provides an alternative to the use of pain management drugs.

Historically women have demanded the Caesarian Section Operation to avoid the pain and trauma of childbirth. The current invention provides an alternative to the use of surgical procedures to remove the baby.

Historically, mothers generally go through screaming, tearing, exhausting, bloody, trauma during labor and delivery. The baby emerges into a bloody, hysterical, crying, screaming, stressful, painful, traumatic, world. The current invention seeks to replace the trauma of childbirth with a loving, comfortable, relaxed welcome into a peaceful world the baby can trust.

This device can be adapted to all mammals.

This device can be adapted to open any orifice.

### PREVIOUS ART

In the past, various patents have issued relating to the assistance of women in childbirth. In particular, U.S. Pat. No. 5,910,146, issued on Jun. 8, 1999, to Alexander teaches a childbirth assisting system utilizing a pliable woven sock placed over the head of the fetus in the birth canal using a system of insertion wands to wrap the head and jaw of the fetus in the way of the novelty item, “Chinese Handcuffs'” and a draw string to tighten the sock around the baby's neck so that the doctor can pull the baby's head and body out of the mother. The current invention allows the baby to effortlessly slide down a tunnel to be born.

U.S. Pat. No. 5,207,687, issued on May 4, 1993, to Bemon teaches a child birth assisting system utilizing a pliable woven basket placed over the head of the fetus in the birth canal using a system of insertion wands to wrap the baby's head, jaw and neck so that the doctor can pull the baby's head and body out of the mother. The current invention eliminates the traction of a baby's head during childbirth.

U.S. Pat. No. 4,597,391, issued on Jul. 1, 1986, to Janko teaches an obstetric tractive device child birth assisting system utilizing a pliable woven basket placed over the head of the fetus in the birth canal using a system of insertion wands to wrap the baby's head, jaw and neck so that the doctor can pull the baby's head and body out of the mother. The current invention reduces the squeezing and trauma that the baby's head goes through during childbirth.

U.S. Pat. No. 1,782,814, issued on Nov. 25, 1930, to Froehlict teaches an obstetrical extractor sling birth assisting system utilizing a using a system of insertion wands and scissors forceps to wrap the baby's neck with a rope so that the doctor can pull the baby's neck, and in turn, head and body out of the mother. The current invention places no ropes around the baby's neck.

U.S. Pat. No. 713,166, issued on Nov. 11, 1902, to St. Cyr teaches a obstetrical tractor child birth assisting system utilizing a pliable woven basket placed over the head of the fetus in the birth canal using a system of insertion wands to wrap the baby's head, jaw and neck so that the doctor can pull the baby's head and body out of the mother. The current invention requires no insertion wands that risk damage the soft skull and sutures of the baby's flexible head.

U.S. Pat. No 497,720, issued on May 16, 1893, to Jones teaches a veterinary obstetrical forceps birth assisting system utilizing a system of insertion wands to wrap the fetus's neck with a rope so that the veterinarian can pull the animal's neck, head and body out of the mother animal. The current invention is an alternative to the use of forceps and ropes tied around the baby's neck.

U.S. Pat. No. 13,453, issued on Aug. 21, 1855, to Buffum teaches an obstetrical instrument child birth assisting system utilizing a pliable woven basket placed over the head of the fetus in the birth canal using a system of insertion wands to wrap the baby's head, jaw and neck so that the doctor can pull the baby's head and body out of the mother. The current invention opens up a clear path for the baby to slide out of which reduces the stress on the baby.

U.S. Pat. No. 3,550,595, issued on Dec. 29, 1970, to Laufe teaches an obstetric tractive device child birth assisting system utilizing a pair of metal forceps or tongs that lock into position on the sides of the baby's head on one end and extend out to form a handle that the doctor uses to drag the baby out of the mother's birth canal. The current invention does not grip the baby's head in any way; instead it opens up a slippery tunnel to slide out of.

U.S. Pat. No. 5,871,499, issued on Feb. 16, 1999, to Hahn teaches a child birth assisting system utilizing a pneumatic girdle to externally augment the secondary force of labor. The current invention requires much less strength and number of contractions than normal to achieve childbirth.

U.S. Pat. No. 5,174,281, issued on Dec. 29, 1992, to Lee teaches a child birth assisting system utilizing a pneumatic girdle to externally augment the secondary force of labor. The current invention needs very little secondary force of labor to achieve childbirth

U.S. Pat. No. 2,597,637, issued on May 20, 1952, to Heidenwolf teaches a child birth assisting system utilizing a pneumatic girdle to externally augment the secondary force of labor. The current invention utilizes a tunnel opened up for the baby to exit the uterus; the secondary force of labor is cut to a minimum which reduces the pain of contractions and the number of contractions.

U.S. Pat. No. 3,794,044, issued on Feb. 26, 1974, to Vennard et al. teaches a child birth assisting system utilizing a pneumatic vacuum cup placed over the head of the fetus to assist the extraction of a baby from the birth canal. The current invention needs no vacuum extraction to suck the baby's head out of the mother.

U.S. Pat. No. 3,765,408, issued on Oct. 16, 1973, to Kawai teaches a child birth assisting system utilizing a pneumatic vacuum cup placed over the head of the fetus to assist the extraction of a baby from the birth canal. The current invention eliminates the obstructions to the movement of the baby's head through the birth canal thereby reducing or eliminating the need for vacuum extraction.

U.S. Pat. No. 3,592,198, issued on Jul. 13, 1971, to Evans teaches a child birth assisting system utilizing a liquid chemical that transforms to a solid chemical that is injected into the birth canal and over the head of the fetus and then thermally or chemically catalyzed or hardened to form a tight mold of durable material around the baby's head to assist the extraction of a baby from the birth canal. The current invention requires no gripping or entrapment of any kind related to the baby's head in order to achieve birth.

U.S. Pat. No. 2,227,673, issued on Jan. 7, 1941, to Price teaches a child birth assisting system utilizing a pneumatic vacuum cup and a pliable woven basket placed over the head of the fetus to assist the extraction of a baby from the birth canal. The current invention eliminates the need for vacuum caps or woven baskets to assist childbirth.

U.S. Pat. No. 2,194,989, issued on Mar. 26, 1940, to Torpin teaches a child birth assisting system utilizing a pneumatic vacuum cup to assist the extraction of a baby from the birth canal. The current invention opens a cylindrical tunnel which reduces the tendency for the baby's head to be squeezed into a banana shape to exit the birth canal. With this reduced pressure, the pneumatic vacuum cup is not necessary for a natural childbirth.

The present invention provides innovative improvements over these examples of previous art and other previous art by introducing a less painful and faster device and method for delivering a baby, which gently and safely opens the birth canal and cervix, within the abdominal cavity, forming an expanding tunnel, which enlarges over a period of time, to become large enough in diameter for the baby's head and body to effortlessly slide out of, for easier, quicker childbirth.

### OBJECTS OF THE INVENTION

The first object of the present invention is to provide an alternative to the pain of childbirth.

The second object of the present invention is to provide an alternative to the exhaustion of childbirth.

The third object of the present invention is to provide an alternative to the use of pain management drugs during childbirth.

The fourth object of the present invention is to provide an alternative to the use of episiotomy vagina cutting operations to remove the baby from a mother's body.

The fifth object of the present Invention is to provide an alternative to the use of the Caesarian Section Operation to remove the baby from a mother's body.

The sixth object of the present invention is to provide a device that opens the cervix and birth canal of a pregnant female within the body cavity instead of within the pinching bones of the pelvis.

The seventh object of the present invention is to provide a device that is inserted into the cervix and birth canal, prior to the baby dropping down into the pelvic bones, allowing a tunnel to be formed so the baby can slide out easily and quickly.

The eighth object of the present invention is to provide a device that opens up the cervix and birth canal forming a tunnel for breach birth delivery or other complications to delivery.

The ninth object of the present invention is to provide a spring loaded or motorized device that opens at a rate that is controlled by a timer mechanism.

The tenth object of the present invention is to provide a device that opens with a ratchet device that can be button disabled to collapse the tunnel after the birth.

The eleventh object of the present invention is to provide a device that alternatively can partially or completely open the cervix and/or birth canal for gynecological exams.

The twelfth object of the present invention is to provide a device that has an enlarging diameter pressure sensing function to allow the device to open at a comfortable rate never exceeding pressure tolerances that might cause a rise in pain or risk of injury.

The thirteenth object of the present invention is to provide a device that has an opening speed that is much slower than the pain tolerance level of the woman's vaginal opening speed as the diameter of the birth canal and the cervix are increased to let the baby slide out.

The fourteenth object of the present invention is to provide a device that can be impregnated or coated with a local anesthetic for application of pain relieving nerve deadening cervix and birth canal surface treatment for less traumatic, less painful birth without epidurals, spinal blocks or other powerful systemic drugs.

The fifteenth object of the present invention is to provide a device with a light source that shines through the clear material making up the device itself, creating an illumination of the entire birth canal for ocular observation by the obstetrician.

The sixteenth object of the present invention is to provide a device with a position retention system to hold the device in place while the function is in progress.

The seventeenth object of the present invention is to provide a device that can open other bodily orifices such as nasal, oral, rectal, wounds, incisions, cavities, punctures, gouges or other surgical or accidental body openings with appropriately sized expandable tunnels for ease of access.

### BRIEF DESCRIPTION OF THE DRAWINGS

FIG. 1 shows a perspective view of an embodiment of the present invention childbirth assisting system illustrating the birth tunnel forming device inserted through the vaginal opening into the birth canal and through the cervix and placed against the baby's head in a closed position (solid lines) and in an open position (dashed lines).

FIG. 2 shows a perspective view of an embodiment of the present invention childbirth assisting system illustrating the birth tunnel forming device in an open position with the baby sliding through the tunnel and being born.

FIG. 3 shows a perspective view of the side of an embodiment of the present invention childbirth assisting system illustrating the birth tunnel forming device in a closed position inserted through the vaginal opening into the birth canal and through the cervix and placed against the baby's head.

FIG. 4 shows a perspective view of the side of an embodiment of the present invention childbirth assisting system illustrating the birth tunnel forming device in an open position with the baby sliding through the tunnel and being born.

FIG. 5 shows a section view of an embodiment of the current invention inserted at 3 cm dilation.

FIG. 6 shows a section view of an embodiment of the current invention opening the birth canal at 5 cm dilation.

FIG. 7 shows a section view of an embodiment of the current invention forming a tunnel in the birth canal at 8 cm dilation.

FIG. 8 shows a section view of an embodiment of the current invention allowing the baby to slide out of the birth canal to be born at 11 cm dilation.

### PREFERRED EMBODIMENT COMPONENT LIST

10 birth tunnel forming device
11 vaginal opening
12 birth canal
13 baby's head
14 pubic bone
15 coccyx
16 cervix
17 dilated cervix
18 anesthetic coating
19 spiral rolled clear tunnel
20 handle
21 finger knob
22 ratchet teeth
23 ratchet release
24 gear drive shaft
25 drive shaft sleeve
26 front drive gear teeth
27 rear drive gear teeth
28 front drive gear holes
29 rear drive gear holes
30 alignment guide slot
31 rolled tunnel
32 unrolled tunnel
33 rolled forward diameter
34 unrolled forward diameter
35 rolled rear diameter
36 unrolled rear diameter
37 forward minimum diameter locking hole
38 rear minimum diameter locking hole
39 vaginal pinching muscle
40 vacuum
41 vacuum tube
42 vacuum manifold drain
43 vacuum manifold intake slots
44 vacuum manifold
45 soft flexible conformal cone
46 light source
47 light source power connector
48 timer motor

### DETAILED DESCRIPTION OF THE EXEMPLARY EMBODIMENT(S)

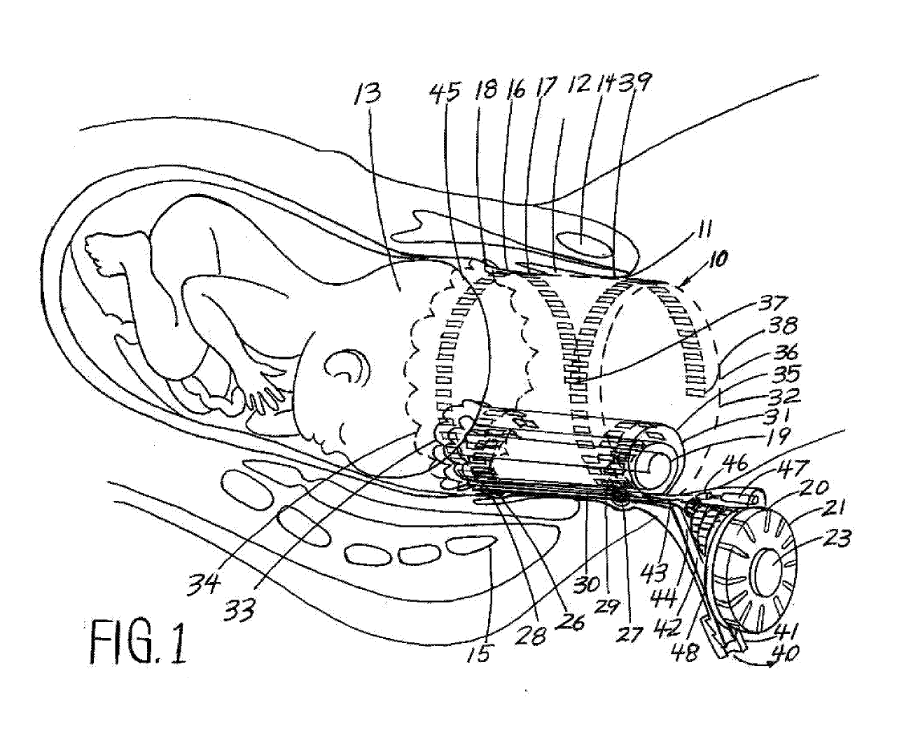

FIG. 1 shows a perspective view of an embodiment of the present invention childbirth assisting system illustrating the 10 birth tunnel forming device inserted through the 11 vaginal opening into the 12 birth canal and placed against the 13 baby's head. The 14 pubic bone in the front and the 15 coccyx bone in the rear define the hard points of the available room for the advancement of the 13 baby's head through the 12 birth canal to be born. The 16 cervix holds the 13 baby's head inside the mother until the mother's water breaks and the 16 cervix opens or dilates slightly to display the 13 baby's head in the opening of the 17 dilated cervix. The rolled 10 birth tunnel forming device is relatively small in diameter as it is inserted into the opening of the 17 dilated cervix and the 10 birth tunnel forming device conforms to the shape of the 13 baby's head through the opening in the 17 dilated cervix. The birth tunnel forming device may include an impregnated or printed 18 anesthetic coating in order to provide local anesthetic pain relief to the parts of the body touching the 10 birth tunnel forming device. This 18 anesthetic coating may be applied or impregnated in layers or encapsulated to provide an anesthetic pain deadening function in a timed release manner to keep the mother comfortable through the birthing process. The 10 birth tunnel forming device has a 19 spiral rolled clear tunnel which is inserted at a small diameter and then is enlarged to form a tunnel that the baby slides out of to be born. The obstetrician controls the movement of the 10 birth tunnel forming device by grasping the 20 handle and guiding the device through the 17 dilated cervix to gently place the device next to the 13 baby's head. In order to start the birthing process, the obstetrician rotates the 21 finger knob, after disengaging the 22 ratchet teeth by depressing the 23 ratchet release on the 20 handle, which causes the rotation of the 24 gear drive shaft within the 25 drive shaft sleeve causing the 26 front drive gear teeth and the 27 rear drive gear teeth to engage and move the 28 front drive gear holes and the 29 rear drive gear holes within the 30 alignment guide slot causing the 31 rolled tunnel to move in a manner that increases the diameter of the spiral resulting in an 32 unrolled tunnel where the 33 rolled forward diameter is enlarged to form the 34 unrolled forward diameter and the 35 rolled rear diameter is enlarged to form the 36 unrolled rear diameter causing the tunnel to expand in the 12 birth canal and increase the diameter of the 17 dilated cervix larger and larger over time to form a tunnel large enough for the baby to slide out of to be born. The 10 birth tunnel forming device is provided with a 37 forward minimum diameter locking hole and a 38 rear minimum diameter locking hole. In order to limit the minimum diameter the 19 spiral rolled clear tunnel may be reduced in size to avoid taking the spiral wound plastic past the point where the elastic memory of the plastic flex capability is exceeded and the plastic will no longer spring out into a tunnel shape again. The 34 unrolled forward diameter is larger than the 36 unrolled rear diameter causing a conical shape to be formed inside the 12 birth canal where the 39 vaginal pinching muscle has a tendency to hold the device inside against the 13 baby's head until the diameter becomes large enough for the baby to slide out of. After the birth, the diameter can be reduced to comfort the mother. The placenta is born through the reduced diameter tunnel. Once everything is out, the diameter of the tunnel is reduced to its minimum, removed and disposed of. The 10 birth tunnel forming device may be provided with a 40 vacuum source in order to remove fluids through a 41 vacuum tube that is connected to a 42 vacuum manifold drain that suctions fluids that collect in 43 vacuum manifold intake slots in the 44 vacuum manifold. The 10 birth tunnel forming device is provided with a flexible 45 soft rounded conformal edge to safely and comfortably expand within the 12 birth canal through the 17 dilated cervix and around the 13 baby's head to afford the greatest protection and comfort to both the baby and the mother. The 10 birth tunnel forming device may be equipped with a 46 light source and 47 light source power connector for the purpose of illuminating the interior of the 12 birth canal for improved ocular acuity during the birthing process. The finger knob and ratchet gear drive system can be upgraded to utilize a 48 timer motor driven opening system. A wind up coil spring driven 48 timer motor can open the tunnel device at a metered rate based on the amount of diameter growth verses discomfort or pain threshold over the time period desired. An electric opening 48 timer motor can be augmented with a pressure sensor to determine the optimal progression of opening speed verses comfort and elasticity of the 12 birth canal and 17 dilated cervix. The 48 timer motor can be electric, spring loaded, pneumatic, hydraulic, vacuum, elastic, gravity or muscle operated. The 48 timer motor may simply be a coiled spring or a v-shaped spring that is compressed and placed in the functional position then allowed to expand against the elastic resistance of the 17 dilated cervix and the 12 birth canal.

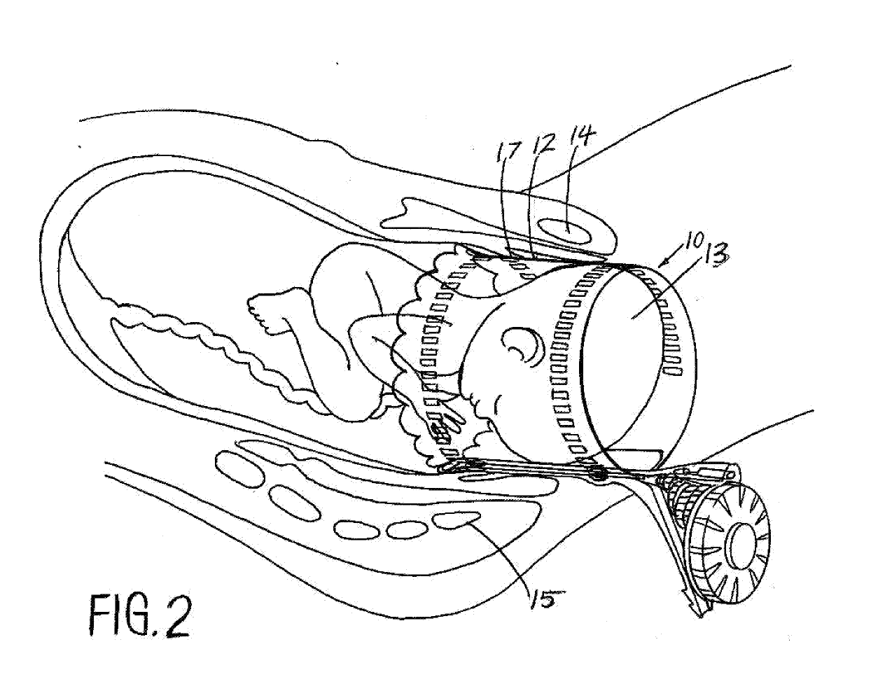

FIG. 2 shows a perspective view of an embodiment of the present invention childbirth assisting system illustrating the 10 birth tunnel forming device enlarged to a diameter that allows the 13 baby's head to slide out of the 17 dilated cervix into the 12 birth canal and out the end of the open tunnel to be born. The 14 pubic bone in the front and the 15 coccyx bone in the rear define the hard points of the available room for the advancement of the 13 baby's head through the 12 birth canal to be born. After the birth, the diameter can be reduced to comfort the mother. The placenta is born through the reduced diameter tunnel. Once everything is out, the diameter of the tunnel is reduced to minimum, removed and disposed of.

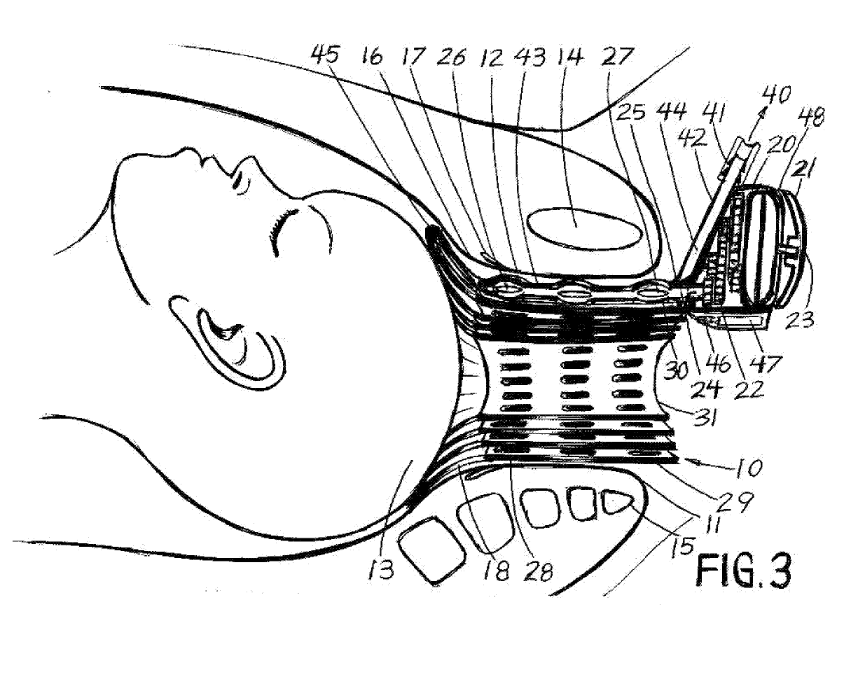

FIG. 3 shows a perspective view of the side of an embodiment of the present invention childbirth assisting system illustrating the birth tunnel forming device in a closed position inserted through the vaginal opening into the birth canal and through the cervix and placed against the baby's head. The 10 birth tunnel forming device is inserted through the 11 vaginal opening into the 12 birth canal and placed against the 13 baby's head. The 14 pubic bone in the front and the 15 coccyx bone in the rear define the hard points of the available room for the advancement of the 13 baby's head through the 12 birth canal to be born. The 16 cervix holds the 13 baby's head inside the mother until the mother's water breaks and the 16 cervix opens or dilates slightly to display the 13 baby's head in the opening of the 17 dilated cervix. The rolled 10 birth tunnel forming device is relatively small in diameter as it is inserted into the opening of the 17 dilated cervix and the 10 birth tunnel forming device conforms to the shape of the 13 baby's head through the opening in the 17 dilated cervix. The birth tunnel forming device may include an impregnated or printed 18 anesthetic coating in order to provide local anesthetic pain relief to the parts of the body touching the 10 birth tunnel forming device. The obstetrician controls the movement of the 10 birth tunnel forming device by grasping the 20 handle and guiding the device through the 17 dilated cervix to gently place the device next to the 13 baby's head. In order to start the birthing process, the obstetrician rotates the 21 finger knob, after disengaging the 22 ratchet teeth by depressing the 23 ratchet release on the 20 handle, which causes the rotation of the 24 gear drive shaft within the 25 drive shaft sleeve causing the 26 front drive gear teeth and the 27 rear drive gear teeth to engage and move the 28 front drive gear holes and the 29 rear drive gear holes within the 30 alignment guide slot causing the 31 rolled tunnel to move in a manner that increases the diameter of the spiral resulting in causing the tunnel to expand in the 12 birth canal and increase the diameter of the 17 dilated cervix larger and larger over time to form a tunnel large enough for the baby to slide out of to be bom. The 10 birth tunnel forming device may be provided with a 40 vacuum source in order to remove fluids through a 41 vacuum tube that is connected to a 42 vacuum manifold drain that suctions fluids that collect in 43 vacuum manifold intake slots in the 44 vacuum manifold. The 10 birth tunnel forming device is provided with a flexible 45 soft rounded conformal edge to safely and comfortably expand within the 12 birth canal through the 17 dilated cervix and around the 13 baby's head to afford the greatest protection and comfort to both the baby and the mother. The 10 birth tunnel forming device may be equipped with a 46 light source and 47 light source power connector for the purpose of illuminating the interior of the 12 birth canal for improved ocular acuity during the birthing process. The 21 finger knob and ratchet gear drive system can be upgraded to utilize a 48 timer motor driven opening system. A wind up coil spring driven 48 timer motor can open the tunnel device at a metered rate based on the amount of diameter growth verses discomfort or pain threshold over the time period desired. An electric opening 48 timer motor can be augmented with a pressure sensor to determine the optimal progression of opening speed verses comfort and elasticity of the 12 birth canal and 17 dilated cervix. The 48 timer motor can be electric, spring loaded, pneumatic, hydraulic, vacuum, elastic, gravity or muscle operated. The 48 timer motor may simply be a coiled spring or a v-shaped spring that is compressed and placed in the functional position then allowed to expand against the elastic resistance of the 17 dilated cervix and the 12 birth canal in order to achieve birth.

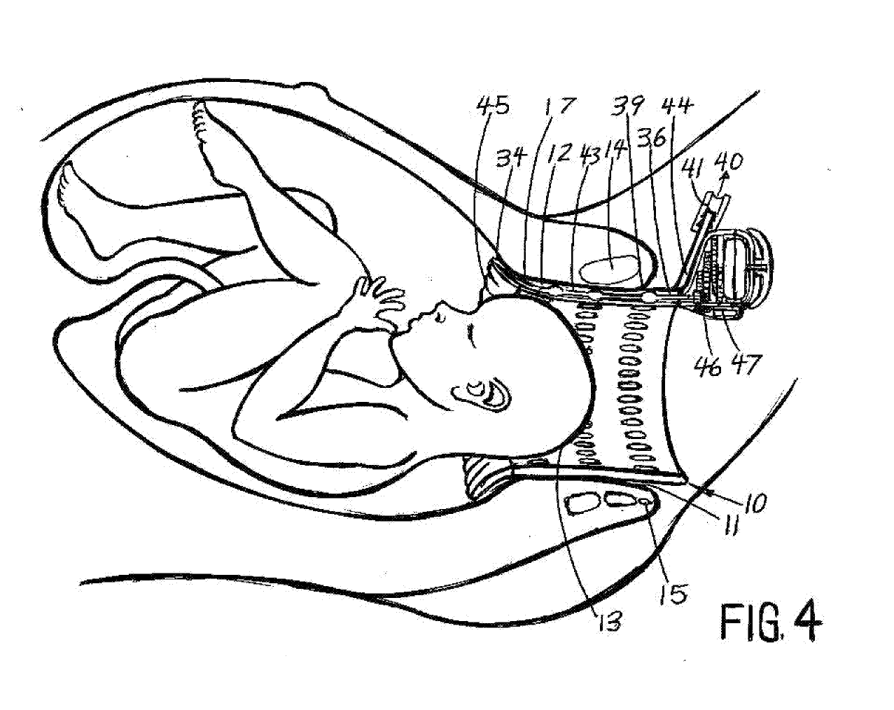

FIG. 4 shows a perspective view of the side of an embodiment of the present invention childbirth assisting system illustrating the birth tunnel forming device in an open position with the baby sliding through the tunnel and being born. The 10 birth tunnel forming-device is inserted through the 11 vaginal opening into the 12 birth canal and placed against the 13 baby's head. The 14 pubic bone in the front and the 15 coccyx bone in the rear define the hard points of the available room for the advancement of the 13 baby's head through the 12 birth canal to be born. The 34 unrolled forward diameter is larger than the 36 unrolled rear diameter causing a conical shape to be formed inside the 12 birth canal where the 39 vaginal pinching muscle has a tendency to hold the device inside for the baby to slide out of. After the birth, the diameter can be reduced to comfort the mother. The placenta is born through the reduced diameter tunnel. Once everything is out, the diameter of the tunnel is reduced to its minimum, removed and disposed of. The 10 birth tunnel forming device may be provided with a 40 vacuum source in order to remove fluids through a 41 vacuum tube that is connected to a 42 vacuum manifold drain that suctions fluids that collect in 43 vacuum manifold intake slots in the 44 vacuum manifold. The 10 birth tunnel forming device is provided with a flexible 45 soft rounded conformal edge to safely and comfortably expand within the 12 birth canal through the 17 dilated cervix and around the 13 baby's head to afford the greatest protection and comfort to both the baby and the mother. The 10 birth tunnel forming device may be equipped with a 46 light source and 47 light source power connector for the purpose of illuminating the interior of the 12 birth canal for improved ocular acuity during the birthing process.

FIG. 5 shows a section view of an embodiment of the current invention inserted at 3 cm dilation. The 10 birth tunnel forming device is placed on the 13 baby's head and the 45 soft flexible conformal cone gently spreads out and slides inside the 17 dilated cervix forming a trumpet bell shape and separating the baby from the mother so that the birthing process can commence.

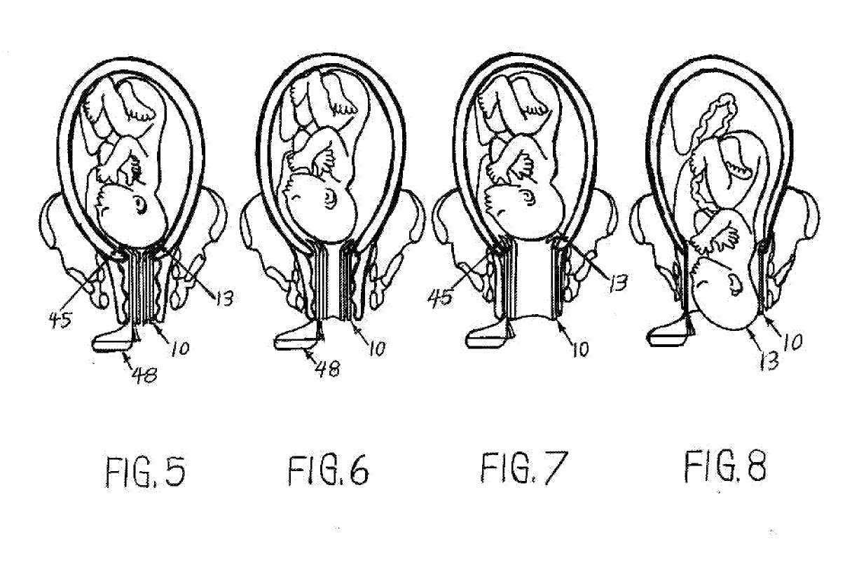

FIG. 6 shows a section view of an embodiment of the current invention opening the birth canal at 5 cm dilation. The 10 birth tunnel forming device starts to enlarge in diameter as the 48 timer motor opens up the diameter and spreads out the cervix.

FIG. 7 shows a section view of an embodiment of the current invention forming a tunnel in the birth canal at 8 cm dilation. The 10 birth tunnel forming device grows larger and larger, wrapping around the 13 baby's head and spreading the 45 soft flexible conformal cone deeper and deeper into the uterus enlarging the size of the tunnel in the 17 dilated cervix.

FIG. 8 shows a section view of an embodiment of the current invention allowing the baby to slide out of the birth canal to be born at 11 cm dilation. The 10 birth tunnel forming device has now opened to a dimension that is larger that the dimension of the 13 baby's head which removes all obstructions and the baby effortlessly slides out to be born.

The preferred embodiments herein disclosed are not intended to be exhaustive or to unnecessarily limit the scope of the invention. The preferred embodiments were chosen and described in order to explain the principles of the present invention so that others skilled in the art may practice the invention. Having shown and described preferred embodiments of the present invention, those skilled in the art will realize that many variations and modifications may be made to affect the described invention. Many of those variations and modifications will provide the same result and fall within the spirit of the claimed invention. It is the intention, therefore, to limit the invention only as indicated by the scope of the claims.

| | | | |
|---|---|---|---|
| 713166 | November, 1902 | St. Cyr | 606/122. |
| 1690942 | November, 1928 | Odell | 606/122. |
| 1782814 | November, 1930 | Froehlich | 606/122. |
| 3139886 | July, 1964 | Tallman et al. | 604/12. |
| 4597391 | July, 1986 | Janko | 606/122. |
| 4875482 | October, 1989 | Hariri et al. | 606/122. |
| 13453 | August, 1855 | Buffum. | |
| 497720 | May, 1893 | Jones. | |
| 713166 | November, 1902 | St. Cyr. | |
| 1448739 | March, 1923 | Godfrey | 128/352. |
| 1690942 | November, 1928 | Odell. | |
| 1782814 | November, 1930 | Froehlich. | |
| 2194989 | March, 1940 | Torpin | 128/361. |
| 2227673 | January, 1941 | Price | 128/352. |
| 2792838 | May, 1957 | Guerriero | 128/361. |
| 3592198 | July, 1971 | Evans | 128/352. |
| 3765408 | October, 1973 | Kawai | 128/352. |
| 3794044 | February, 1974 | Vennard et al. | 128/352. |
| 4018230 | April, 1977 | Ochiai et al. | 128/344. |
| 4136679 | January, 1979 | Martinez et al. | 128/1. |
| 4512347 | April, 1985 | Uddenberg | 128/352. |
| 4597391 | July, 1986 | Janko | 128/361. |
| 4602623 | July, 1986 | Cherkassky | 128/323. |
| 13453 | August, 1855 | Buffum | 606/122. |
| 713166 | November, 1902 | St. Cyr. | |
| 1690942 | November, 1928 | Odell. | |
| 1782814 | November, 1930 | Froehlich. | |
| 2618272 | November, 1952 | Larson | 606/122. |
| 3139886 | July, 1964 | Tallman et al. | |
| 3550595 | December, 1970 | Laufe. | |
| 3605748 | September, 1971 | Salinas-Benavides | 128/323. |
| 3665925 | May, 1972 | Dersookian | 128/323. |
| 3785381 | January, 1974 | Lower et al. | 128/323. |
| 3789849 | February, 1974 | Laufe et al. | 128/323. |
| 3794044 | February, 1974 | Vennard et al. | 128/352. |
| 4597391 | July, 1986 | Janko | 128/361. |
| 4875482 | October, 1989 | Hariri et al. | 128/352. |
| 5122148 | June, 1992 | Alexander | 606/122. |
| 5207687 | May, 1993 | Bernon | 606/122. |
| 5217467 | June, 1993 | Alexander | 606/122. |
| 5318573 | June, 1994 | Alexander | 606/122. |
| 5593413 | January, 1997 | Alexander | 606/122. |
| 5632750 | May, 1997 | Alexander et al. | 606/122. |
| Re31158 | February, 1983 | Carter et al. | |
| 2597637 | August, 1952 | Heidenwolf. | |
| 4294261 | October, 1981 | Baker et al. | |
| 4321929 | March, 1982 | Lemelson et al. | |
| 4520820 | June, 1985 | Kitchin et al. | |
| 4531516 | July, 1985 | Poole et al. | |
| 4534338 | August, 1985 | Crosbie et al. | |
| 4548198 | October, 1985 | Manes. | |
| 4736731 | April, 1988 | Van Patten. | |
| 4815472 | March, 1989 | Wise et al. | |
| 4873986 | October, 1989 | Wallace. | |
| 4949730 | August, 1990 | Cobben et al. | |
| 5013396 | May, 1991 | Wise et al. | |
| 5113868 | May, 1992 | Wise et al. | |
| 5174281 | December, 1992 | Lee. | |
| 5218972 | June, 1993 | Gorsuch et al. | |
| 5247932 | September, 1993 | Chung et al. | |
| 5343064 | August, 1994 | Spangler et al. | |
| 5377524 | January, 1995 | Wise et al. | |
| 5383893 | January, 1995 | Daneshvar. | |
| 5405356 | April, 1995 | Hahn et al. | |
| 5464420 | November, 1995 | Hori et al. | |
| 5558638 | September, 1996 | Evers et al. | |
| 5566680 | October, 1996 | Urion et al. | |
| 5576251 | November, 1996 | Garabedian et al. | |
| 5578843 | November, 1996 | Garabedian et al. | |
| 5645563 | July, 1997 | Hahn et al. | |

1. A birth assisting device that is inserted into the birth canal in the form of a cylindrical spiral roll of flexible durable material that passes from the opening of the birth canal through the cervix to rest on the baby's head in the uterus, then the spiral roll is unrolled causing an enlarging hollow tunnel to form in the birth canal and the cervix until the tunnel is large enough for the baby's head and body to slide through to be born.

2. The birth assisting device of claim 1 with one or a plurality of the following features or functions; lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

3. A birth assisting device placed inside the birth canal that transforms from a small diameter cylinder to a large diameter hollow tunnel that allows the baby to slide through the tunnel to be born.

4. The birth assisting device of claim 3 with one or a plurality of the following features or functions; lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

5. A birth assisting device placed inside the birth canal that transforms from an easy to insert shape to a hollow tunnel that allows the baby to slide through the tunnel to be born.

6. The birth assisting device of claim 5 with one or a plurality of the following features or functions; lighting, light guides, fiber optic lighting, LED lighting, luminescence, electric lighting, motorized opener, spring opener, timer opener, gear drive opener, friction drive opener, pneumatic opener, hydraulic opener, lever opener, screw opener, spiral opener, cam opener, ratchet opener, ratchet, vacuum, vacuum fluid removal, vacuum vapor removal, laser, anesthetic, timed release anesthetic, lubrication, pressure sensor, variable speed opening, pressure controlled opening speed, mother controlled opening speed, pain sensing opening speed control, position retention system, camera, video camera, lenses, variety of lengths and diameters, sized to open any sized orifice, soft edges, flexible edges, stiff edges, handle, ergonomic handle, cylindrical tunnel, conical tunnel, anthropomorphic tunnel, curved tunnel, flexible tunnel, cylindrical applicator, conical applicator, conical cylinder, curved cylinder, flexible cylinder, conformal cylinder, anthropomorphic cylinder, cylinder with rounded tip, cylinder with flexible tip, cylinder with a soft tip, lock, safety lock, limited slip clutch, clutch, pressure sensing clutch, plastic components, metal components, composite components, paper components, mesh components, recyclable components, sterile components, reusable components, transparent components, translucent components, opaque components, anti-glare components, glow-in-the-dark components, anesthetic components, anesthetic embedded components, anesthetic impregnated components, anesthetic coated components, lubrication coated components and/or a squeeze actuated tunnel enlargement ratchet lock handle.

7. A birth assisting device of claim 5 adapted to all mammal's birth canals.

8. A birth assisting device of claim 5 adapted to all animal's birth canals.

9. A birth assisting device of claim 5 adapted to open an incision and become a retractor to hold tissue open during an operation.

10. A birth assisting device of claim 5 adapted to open up the cervix and birth canal forming a tunnel for breach birth delivery.

11. A birth assisting device of claim 5 with a spring loaded or motorized device that opens the cervix and birth canal at a rate that is controlled by a timer mechanism.

12. A birth assisting device of claim 5 with a device that opens with a ratchet device that can be button disabled to collapse the tunnel after the birth.

13. A birth assisting device of claim 5 with a device that alternatively can partially or completely open the cervix and/or birth canal for gynecological exams.

14. A birth assisting device of claim 5 with a device that has an enlarging diameter pressure sensing function to allow the device to open at a comfortable rate never exceeding pressure tolerances that might cause a rise in pain or risk of injury.

15. A birth assisting device of claim 5 with a device that has an opening speed that is much slower than the pain tolerance level of the woman's vaginal opening speed as the diameter of the birth canal and the cervix are increased to let the baby slide out.

16. A birth assisting device of claim 5 with a device that can be embedded, impregnated or coated with a local anesthetic for application of pain relieving nerve deadening cervix and birth canal surface treatment for less traumatic, less painful birth without epidurals, spinal blocks or other powerful systemic drugs.

17. A birth assisting device of claim 5 with a device with a light source that shines through the clear material making up the device itself, creating an illumination of the entire birth canal for ocular observation by the obstetrician.

18. A birth assisting device of claim 5 with a device with a position retention system to hold the device in place while the function is in progress.

19. A birth assisting device of claim 5 adapted as a device that can open other bodily orifices such as nasal, oral, rectal, wounds, incisions, cavities, punctures, gouges or other surgical or accidental body openings with appropriately sized expandable tunnels for ease of access.
